(19) United States
(12) Patent Application Publication
(10) Pub. No.: US 2004/0064057 A1
(43) Pub. Date: Apr. 1, 2004

(54) Measuring circulating blood volume through retinal vasculometry

(75) Inventor: Andrew Siegel, Arlington, MA (US)

Correspondence Address: TESTA, HURWITZ & THIBEAULT, LLP, HIGH STREET TOWER, 125 HIGH STREET, BOSTON, MA, 02110, US

(73) Assignee: Massachusetts Institute of Technology, Cambridge, MA

(21) Appl. No.: 10/632,212

(22) Filed: Jul. 31, 2003

Related U.S. Application Data
(60) Provisional application No. 60/399,826, filed on Jul. 31, 2002.

(51) Int. Cl. A61B 5/02

(52) U.S. Cl. 600/500

(57) ABSTRACT

This invention relates to a system, method and apparatus for assessing a patient's risk of cardiovascular collapse by measuring circulating blood volume through transpupillary measurement of retinal vasculature. The system is useful for predicting patients at risk of suffering hypovolemic shock, cardiogenic shock, anaphylactic shock, or septic shock. The system includes a light source, an optical assembly, an imaging device, a processor, and an output device. Images of the eye are obtained and analyzed using a software package. The measurements obtained are compared to a database to determine if the patient is at risk of suffering vascular collapse.

### CROSS-REFERENCE TO RELATED APPLICATIONS

This application is based on and claims priority to U.S. provisional patent application serial No. 60/399,826, filed Jul. 31, 2002.

### STATEMENT REGARDING FEDERALLY SPONSORED RESEARCH

This invention was supported under U.S. Air Force Grant F19628-00-C-0002.

### TECHNICAL FIELD

The present invention relates to a system, apparatus and method for assessing the blood volume of a patient, more specifically to assess blood volume by retinal vasculometry.

### BACKGROUND OF THE INVENTION

Under normal circumstances, the body maintains a delicate balance between the circulating blood volume and the volume within the entire cardiovascular system. This is necessary to provide sufficient venous return to feed the hydraulic demand of the heart. Since the heart has evolved to operate as a positive or neutral pressure pump, it requires a steady supply of blood to the vena cava. In order to maintain this supply, the body employs a number of physiologic feedback loops to both maintain adequate circulating blood volume and to match the vascular volume to this blood volume, thus maintaining adequate venous return.

The physiological responses the body uses to compensate for blood loss are rate dependent. At very slow rates of blood loss, volume replacement occurs through reduced renal fluid excretion and increased hematopoesis. At moderate loss rates, the reduction in capillary pressure results in diffusion of lymph and intracellular fluid back into the vascular compartment. If the rate of blood loss is too rapid to be replaced through hematopoesis or fluid diffusion, volume compensation is achieved through vascular constriction. If blood loss continues, this compensatory vascular constriction process will eventually fail, at which point the patient suffers from hypovolemic shock. Hypovolemic shock refers to a medical condition in which the heart is unable to supply enough blood to the body because of blood loss, circulatory failure, or inadequate blood volume.

When there is rapid blood loss, medical staff are generally alerted by overt symptoms which are easy to detect including hypotension, tachycardia, and tachypnea and the patient is treated immediately. However, when blood is lost at an intermediate rate, as described above, from an undetected injury, symptoms are much more difficult to detect. Casualties with slow internal bleeding may exhibit normal vital signs despite the loss of as much as half of their circulating blood volume. In particular, young and healthy patients usually exhibit few, if any, overt symptoms until they approach vascular collapse.

Even with vigorous resuscitative efforts, such as fluid replacement and drug therapy, the amount of tissue ischemia which results both during and after acute vascular collapse generally leads to tissue necrosis and eventual death. If a patient is not under direct observation in an acute care setting (which is unlikely for casualties during a combat situation or with accident victims), the signs of impending vascular collapse may be missed, with potentially tragic consequences. If significant blood loss can be detected prior to vascular collapse, resuscitative measures can be taken promptly, thereby reducing mortality in these patients.

It is, therefore, an object of the invention to provide a means of measuring circulating blood volume to help detect patients at risk of hypovolemic shock.

### SUMMARY OF THE INVENTION

The invention described herein provides a system, method and apparatus for measuring circulating blood volume through transpupillary measurement of retinal vasculature. The invention is useful for determining whether trauma victims and casualties suffering from internal or external blood loss are at risk of vascular collapse, cardiogenic shock, anaphylactic shock, or septic shock.

One aspect of this invention provides a system for assessing the blood volume of a patient. According to one embodiment of the invention, the system includes an imaging device for capturing images of the patient's retina as pixel data. The system may feature a processor in communication with the imaging device. According to one embodiment, the processor compares the pixel data to a database to determine if the patient is at risk of vascular collapse.

The system in various embodiments has the following features. In one embodiment, the processor measures a vasculature characteristic from the pixel data. In another embodiment, the processor measures non-vascular tissue from the pixel data. In another embodiment, the vascular measurement comprises measurements of arterial vessel diameter. In another embodiment, the vascular measurement comprises measuring venous vessel diameter. In yet another embodiment, the vascular measurement comprises measurements of arterial and venous vessel diameters. After obtaining measurements of arterial and venous vessel diameters, in one embodiment, a ratio of venous diameter to arterial diameter is calculated from the pixel data, and the ratio is compared to the database to determine if the patient is at risk of vascular collapse.

In one embodiment, the imaging device captures images of the retina from a region around the patient's optical disk. In another embodiment, the pixel data is obtained from a user-defined area on the retina. In other embodiments, the user-defined area is toroidal or circular in shape.

In another embodiment, the imaging device includes a CCD-based camera for capturing images of the patient's retina. Alternatively, images of the patient's retina are captured using a MOS based camera. In another embodiment, the imaging device includes a single element detector. In other embodiments according to the invention, the processor outputs an alert if the measurements are below or above a predetermined range of values. In other embodiments, the processor distinguishes between vascular and non-vascular tissues, or between arterial vessels, venous vessels, and non-vascular tissues. In yet another embodiment, the system includes an output device. In certain embodiments, the output device is selected from a laptop monitor, a desktop computer monitor, a television screen, a PDA, a printing device, and a pager.

In other embodiments, the system includes a light source, and/or an optical assembly. In yet another embodiment, the light source is selected from a light emitting diode, an incandescent light bulb, a flash lamp, and a laser diode. In another embodiment, the system is portable.

In further embodiments according to the invention, the data is captured at a center wavelength in the range of about 400 nm to about 1000 nm. In another embodiment, the data is captured at a center wavelength in the range of about 500 nm to about 700 nm. In another embodiment, the light source provides light having a center wavelength in the range of about 400 nm to about 1000 nm. In another embodiment, the light source provides light having a center wavelength in the range of about 500 nm to about 700 nm.

In another aspect of the invention, a method is provided for assessing the blood volume of a patient. The method includes capturing images of the patient's retina as pixel data using an imaging device and using a processor to calculate measurements of retinal vessels from the pixel data. In another step, the calculated measurements are compared with a database to determine if the patient is at risk of vascular collapse.

The method may also include the step of outputting an alert if the measurements are below or above a predetermined range of values. In addition the method may also include the step of using spectrometry to distinguish arterial vessels, venous vessels, and non-vascular tissue. In a further adaptation, the step of comparing the calculated measurements with a database to determine if the patient is at risk of vascular collapse further includes using a database including patient specific data obtained from the patient before or after the injury, or data from individuals with a known risk of vascular collapse.

These and other objects, along with advantages and features of the present invention herein disclosed, will become apparent through reference to the following description, the accompanying drawings, and the claims. Furthermore, it is to be understood that the features of the various embodiments described herein are not mutually exclusive and can exist in various combinations and permutations.

### BRIEF DESCRIPTION OF THE DRAWINGS

In the drawings, like reference characters generally refer to the same parts throughout the different views. Also, the drawings are not necessarily to scale, emphasis generally being placed upon illustrating the principles of the invention. In the following description, various embodiments of the present invention are described with reference to the following drawings, in which:

FIG. 1 depicts a schematic view of a system for measuring retinal vasculature according to an illustrative embodiment of the invention.

FIG. 2 depicts a schematic view of a system for measuring retinal vasculature according to another illustrative embodiment of the invention.

FIG. 3 depicts a schematic view of a system for measuring retinal vasculature according to another illustrative embodiment of the invention.

FIG. 4 is a photographic image of the eye being analyzed using a custom software package.

FIG. 5A is a photographic image of the retina including retinal arteries and veins as captured by the imaging device according to an illustrative embodiment of the invention.

FIG. 5B is an enlargement of the photographic image in FIG. 5A.

FIG. 6A is a photographic image of the retina including arteries and veins in a normal animal obtained by a system for measuring retinal vasculature including a digital camera according to an illustrative embodiment of the invention.

FIG. 6B is a photographic image of the retina including arteries and veins in a normal animal in which 32% of the blood volume has been removed, obtained by a system for measuring retinal vasculature including a digital camera according to an illustrative embodiment of the invention.

FIGS. 7A-7C are tables showing the results of the first, second and third rodent experiments respectively.

### DETAILED DESCRIPTION

The present invention provides a system of monitoring the volume of circulating blood within the vasculature and can be used to detect patients that are at risk of vascular collapse, cardiogenic shock, anaphylactic shock, or septic shock. For instance, the proposed technology may find use in the military to help diagnose and triage combat victims. Civilian applications may include analyzing victims at the scene of an accident and rapidly screening ER patients suffering from penetrating wounds or blunt trauma. Other uses may include monitoring postoperative patients for blood loss due to inadequate hemostasis, torn stitches, etc. The invention may also aid in the recovery of severe burn patients, who often require constant fluid management to prevent dehydration, and for whom standard measurements, such as monitoring urine output, might be difficult or impossible to perform. Since the cost of providing chronic care for those surviving an episode of multiple organ dysfunction is often quite high, there may be significant interest from HMOs in employing this technology for economic reasons as well.

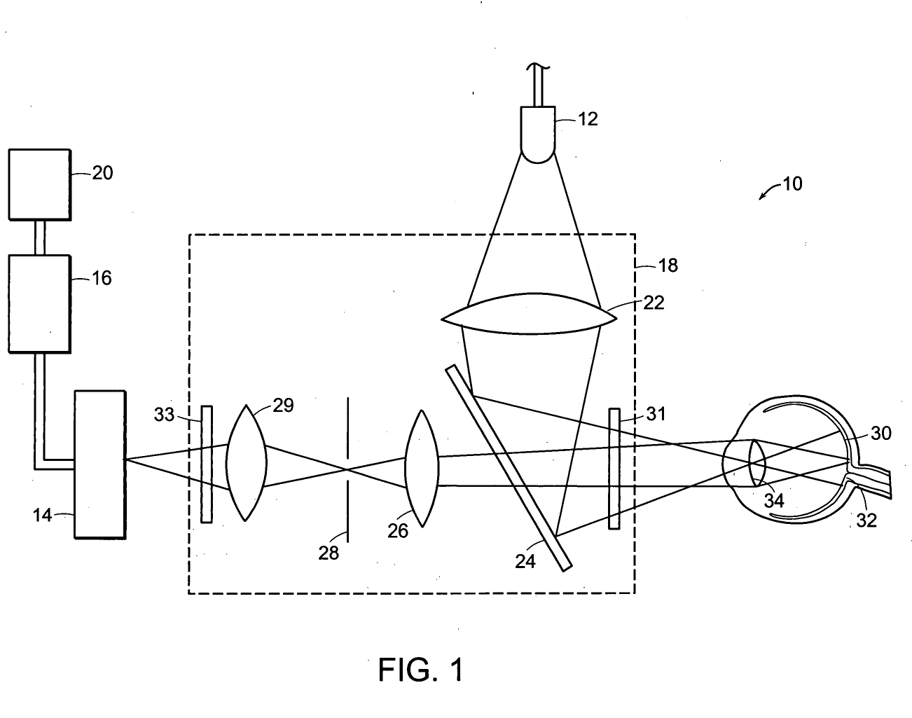

FIG. 1 depicts a schematic view of a system 10 for assessing blood volume in a patient according to an illustrative embodiment of the invention. According to the illustrative embodiment, the system 10 includes a light source 12, an imaging device 14, a processor 16 which is in communication with the imaging device 14, an optical assembly 18, and an output device 20.

According to one feature of the invention, the light source 12 is provided to illuminate the eye, for example, particularly the retina 30. In a particular embodiment, the entire optic disk 32 is illuminated. According to the illustrative embodiment, the light source 12 is one or more light emitting diodes (LEDs) having a wavelength anywhere in the visual spectrum. In an alternative embodiment, the light source 12 is an incandescent light bulb that is filtered to provide a specific wavelength of light to the eye. In yet another embodiment, the light source 12, for example, is a flash lamp or one or more laser diodes. In general, any type of light source may be used to illuminate the eye and the light source 12 is not limited to those described.

According to the illustrative embodiment, any wavelength of light may be used. In a particular embodiment, wavelengths from about 400 nm to about 1000 nm, preferably in the range of 500 nm to about 700 nm are utilized to generate visual contrast in the light reflected from arteries, veins and non-vascular tissue. In one feature of the illustrative embodiment, particularly when a broad spectrum of light is utilized, a bandpass filter may filter light to generate the desired wavelength or wavelength range before the light is directed onto the eye. In another embodiment, a broad spectrum of light is directed onto the eye, and a filter is placed after the optical assembly 18 to generate the desired wavelength or wavelength range before the light is directed onto the imaging device 14. In embodiments of the invention including a laser or an LED light source, a filter is generally not required.

With continued reference to FIG. 1, the illustrated embodiment includes the optical assembly 18 which includes a first lens 22, a second lens 26, a third lens 29, a beam splitter 24, a limiting aperture 28, and a set of polarizers 31, 33. The polarizers 31, 33 may be linear or circular polarizers. According to the illustrative embodiment, the light generated by the light source 12 is collimated by the first lens 22 and is directed by the beam splitter 24 through the polarizer 31, the polarizer 31 being positioned near to the eye, for instance from about 0.5 inches to about 3.0 inches away from the eye. The light then travels through the lens of the eye 34 onto the retina 30. After the light reflects off the retina 30, it passes through the lens of the eye 34 and the polarizer 31, the polarizer 31 reducing the glare from the image reflected from the retina 30. The reflected light then passes through the second lens 26, the third lens 29 and the polarizer 33, the polarizer 33 being positioned near the imaging device 14 or coupled to it to reduce glare before an image is captured by the imaging device 14. The third lens 29 forms the conjugate image of the retina onto the imaging device 14. According to one feature of the illustrative embodiment, the limiting aperture 28 including, for example, a small hole, and, optionally, the third lens 29 and polarizer 33 are provided to filter stray light. The second lens 26 and third lens 29 can also function to compensate for patient specific refractive and accommodation errors, for instance, where the patient is focusing and whether the patient is near or far sighted.

According to the illustrative embodiment, the imaging device 14 is a multi-based element detector, for example, a Charge Coupled Device (CCD) based sensor or a Metal Oxide Semiconductor (MOS) based sensor, or alternatively a single element detector. According to one feature, a recording of the eye such as the retina may be obtained using a digital camera, for example. In another embodiment, a single element detector, such as the UV-100 provided by EG&G (Gaithersburgh, USA), is used to measure the intensity of light reflected from a specified region of the retina to determine blood volume. In another embodiment, blood volume can be determined by 3-D data obtained, for example, by optical tomography. It will be appreciated that any other type of imaging device 14 may be used in accordance with the invention, however, it is preferable that the imaging device 14 is a digital camera or digital video recorder or other solid-state detector, so that the time consuming step of developing film is avoided.

According to another embodiment of the invention, a processor 16 is included in the apparatus 10 to store the images captured by the imaging device 14. For instance, the pixellated image data can be stored in an array and processed using MATLAB® software provided by The MathWorks, Inc. (Natick, Mass.). Alternatively, the images can be stored in any other appropriate format in accordance with the invention. In one embodiment, the processor 16 may analyze the images using available commercial software, for instance MATLAB®. In other embodiments, the public domain NIH Image program (developed at the U.S. National Institutes of Health and available on the Internet at http://rsb.info.nih.gov/nih-image) may be used to analyze the image or the GNU Image Manipulation Program “The Gimp” (available on the Internet at http://www.gimp.org/the_gimp.html) may be used. In yet another embodiment, custom designed software may be used to analyze the captured image of the retina 30.

As an example, according to a preferred embodiment of the illustrative invention, the processor 16, running the NIH Image program, performs several functions. First, the processor 16, acting as a spectrometer, analyzes a first image obtained with a light source 12, for example emitting light having a center wavelength about 680 nm. In this embodiment, the light source 12 provides spectral resolution. Alternatively, the image may be obtained using a light source 12 with a broad spectrum, such as a flash lamp, and the reflected light from the retina can be filtered by a bandpass filter, for example a 680 nm bandpass filter, placed in front of the imaging device 14. In this embodiment, the filter provides spectral resolution. The processor 16 using NIH Image can then distinguish arteries, veins and non-vascular tissue through contrast, since light reflected from arteries appears lighter than light reflected from veins when using a source with a center wavelength of 680 nm. In another embodiment, a second light source 12 (not shown), for example, having a center wavelength of about 520 nm is directed onto the eye or a broad spectrum light source 12 is used and filtered with a 520 nm bandpass filter placed in front of the imaging device 14 as described above. The processor running NIH Image then analyzes the image captured from this setup, and can locate the boundaries between vascular and non-vascular tissue as a result of the high contrast between these tissues at this wavelength. In one embodiment, the data from the image captured with a source wavelength of 680 nm is used together with the image captured with a source wavelength of 520 nm to more accurately distinguish between arteries, veins, and non-vascular tissue. This information assists in obtaining measurements of arterial and venous vessel diameter, as described below.

As another function, the processor 16 measures a vascular characteristic from the pixel data. For instance, in one embodiment, the processsor 16 measures the diameter of at least one retinal artery and retinal vein, and establishes a ratio of vein diameter to artery diameter. The ratio of veins to arteries, along with the average of the vessel diameters, is compared to a database having, for example, similar vessel data obtained from the patient prior to the injury or, as another example, vessel data obtained after the injury. An assessment is then made whether the patient is at risk of vascular collapse. As an alternative, the vessel diameter data is compared to a database of ratios of individuals with a known risk of vascular collapse, and an assessment is made whether the patient may suffer vascular collapse.

According to the illustrative embodiment depicted in FIG. 1, the system 10 may feature an output device 20 coupled to the processor 16. The output device 20 may be a laptop monitor, a desktop computer monitor, a television screen, a PDA or any other visual or acoustic device suitable for viewing the output from the processor 16. In another embodiment, the output of the processor 16 may be directed to a printing device 20 or any other device 20, such as a pager 20, that may alert a professional of a medical emergency.

According to another feature of the illustrative invention, the light source 12, the optical assembly 18, and the imaging device 14 are combined into a single device. Alternatively, a device may include the light source 12, the optical assembly 18, the imaging device 14, as well as the display 20. Such devices have been developed by Retinal Technologies LLC (Winchester, USA) and by Nidek Co. Ltd., such as the Nidek NM-100D (Nidek Co., Ltd., Gamagori, Japan). Alternatively, in yet another embodiment, a device may include the light source 12, the optical assembly 18, the imaging device 14, the processor 16, as well as the display 20. It will be appreciated that many combinations of the system 10 components are possible all of which are in accordance with the invention. Moreover, it will also be appreciated that each embodiment of the system 10 can be manufactured to be rugged and easily portable, for instance in a handheld device, or alternatively can be designed for use in a hospital setting where the system 10 is not easily transported.

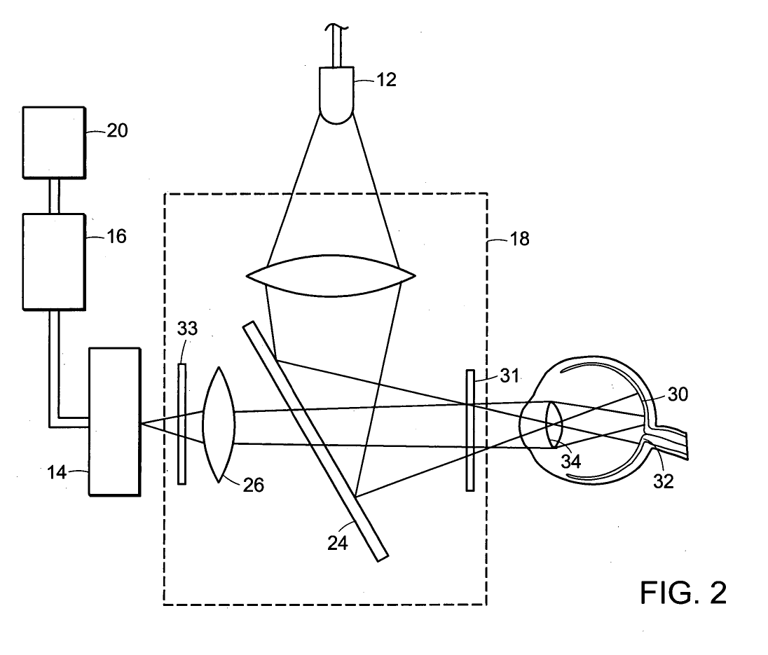

FIG. 2 depicts a schematic view of a system for measuring retinal vasculature according to another illustrative embodiment of the invention. According to the illustrative embodiment, if the imaging device 14 does not have an internal optical assembly 18, the imaging device 14 may be placed after the second lens 26. Alternatively, the imaging device 14 may be placed after the limiting aperture 28, the third lens 29 and polarizer 33 (not shown) depending on the need to filter the image. Filtering the image may be particularly useful if a diffuse light source 12 such as an incandescent bulb or a strobe lamp is utilized. If the imaging device 14 includes an internal optical assembly 18, then the imaging device 14 may be placed in front of the eye without the use of a further external optical assembly 18. The image captured by the imaging device 14 is stored in memory either in the imaging device 14 itself, or in an external device, such as a processor 16.

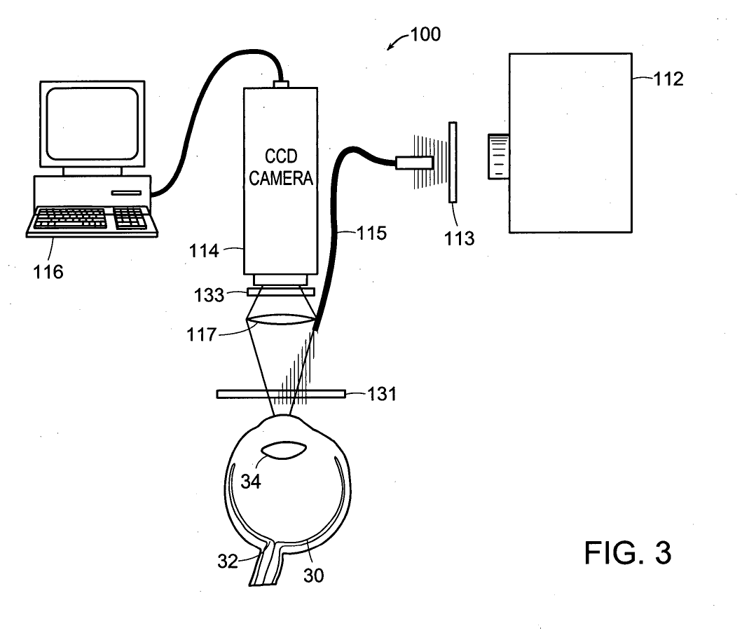

FIG. 3 is a schematic view of a system 100 for measuring retinal vasculature according to another embodiment of the invention. The system 100 illustrated in FIG. 3 generally functions as described above. The system 100 includes a Mercury-Xenon lamp light source 112. The light is filtered with a 570 nm bandpass filter 113 before being supplied to the optical disk region 32 of the eye using a fiber optic cable 115. A polarizer 131 is used to eliminate glare from light reflecting from the eye. A conjugate image of the reflected light is obtained using a lens 117, and the image is captured by a CCD camera 114, which may be coupled to a polarizer 133. The images captured may be analyzed using a processor 116 running, for example, MATLAB® software. Using this software, arteries, veins and nonvascular tissue may be distinguished using spectroscopy as described above and measurements of vasculature may be obtained from the pixel data.

According to a preferred embodiment of the invention, optical measurements are made of the retinal vasculature, particularly around the optic disk 32, to determine vascular volume. The vascular volume is measured as described herein and correlated with the patient's blood volume to determine whether the patient is at risk for cardiovascular collapse. Measuring vasculature of the retina 30 is advantageous compared to measuring vasculature at other sites within the body, for instance, the mouth, legs, or intestines, for several reasons. For example, since the retina 30 is located deep within the skull and follows the temperature of the cerebral cortex quite closely, a significant degree of immunity to environmental conditions or states of arousal exists at the retina 30. Factors that normally affect blood flow to the periphery of the body, such as heat, cold, physical exertion, emotion, etc. do not impact blood flow to the retina 30 to the same extent. For instance, if the legs of a person become chilled, the vessels in the legs will experience vasoconstriction to preserve core temperature. Similarly, if an individual's legs are exposed to heat, the vessels in the legs will dilate. Therefore, measuring vasculature in the legs, or other extremities, to determine whether an individual is experiencing hypovolemic shock may lead to erroneous conclusions because of confounds. Since the vessels in the retina 30 are subject to fewer confounds than vessels at the periphery of the body, the retina 30 is a preferred location for obtaining estimates of blood volume loss through vascular measurement.

The retina 30 is also a preferred location for measuring vascular volume because the vessels in the region are less impacted by stress than are peripheral vessels. Stress causes the body to increase the levels of catecholamines and other vasoactive substances in the blood, which can affect both the arterial and venous vasculature. Therefore, measuring vasculature at a location other than the retina 30 or cerebral vasculature when the body is experiencing stress may lead to a false conclusion the body is experiencing blood volume loss, when in reality the stress is causing vasoconstriction.

Another reason that retinal vasculometry is a preferred method for measuring blood volume is that circulation within the eye is governed by the same cerebral autoregulatory mechanisms as the brain itself. This means that both retinal metabolic rate and retinal perfusion remain constant over a wide range of arterial blood pressure, up to the point of vascular collapse in healthy individuals. Measuring vasculature at the retina 30 is therefore a good indicator of blood volume loss because like the brain, the retina 30 consumes a large amount of oxygen, and when the systemic blood pressure decreases, the arterial vasculature in the eye, specifically pre-capillary arterioles, will dilate as needed to maintain adequate perfusion, thus reducing confounds due to changes in blood pressure.

Yet another advantage of determining blood volume by retinal vasculometry relates to the intraocular pressure in the eye. Since the intraocular pressure in the eye is greater than ambient, the pressure within the eye may enhance the vasoconstriction seen in the venous vasculature, especially with reduced blood volume from hypovolemia. Since the average venous perfusion pressure is already relatively low compared to arteries, a reduction in venous backpressure should create a greater pressure differential, and hence a larger vasoconstrictive effect within the eye than in surrounding tissue, thereby making vasoconstriction simpler to detect.

A final advantage of measuring retinal blood vessels is that observations of vasculature can be made noninvasively, painlessly, and with minimal physical contact with the patient. The transparent and refractive properties of the eye make it ideal for performing optical measurements of the retina 30 without the need to resort to more invasive sensing techniques. Moreover, biometric devices to image the retina with minimal or no contact with the patient already exist.

In another aspect, the invention provides a method for measuring blood volume to detect a patient at risk of vascular collapse. With reference to FIG. 1, a medical professional may use the illustrative system 10 according to the following exemplary steps. As a first step, the medical professional selects a light source 12 to be used in obtaining a first image, the light source having a center wavelength between about 400 nm to about 1000 nm. Preferably, a light source 12 with a center wavelength between about 500 nm to about 700 nm is selected, since with these wavelengths there is sufficient contrast in the reflected light from the eye to distinguish arteries, veins, and non-vascular tissue through spectroscopy. As an additional component of the first step, a light source having a wavelength between about 400 nm to about 500 nm may be used to obtain a second image. At these wavelengths of light, there is sufficient contrast for the processor 16 running, for example, NIH Image to distinguish the boundaries of retinal vasculature from non-vascular tissue through spectroscopy as described above. It will be appreciated that in cases where the light source 12 is combined with the imaging device 14, it may be unnecessary to provide an additional external light source.

As an exemplary second step, the medical professional collimates the light emanating from the light source 12 using a first lens 22, and directs the light through a polarizer 31 and through the lens of the eye 34, preferably onto the optic disk region 32 of the retina 30, using a beam splitter 24. After the light reflects off the retina 30 and travels through the lens of the eye 34 and the polarizer 31, the medical professional as a third step places a second lens 26 in the path of the reflected light to form the conjugate image of the retina. In the fourth step, the medical professional passes the reflected light through a limiting aperture 28. Optionally, the reflected light passes through a third lens 29 and/or a polarizer 33 to further improve image contrast by removing unwanted light rays. The third lens 29 also functions to eliminate patient specific characteristics, for instance, where they are focusing. In an alternative embodiment of the method, steps two through four may be eliminated if the optical assembly 18 is incorporated into a single device, such as the Nidek NM-100D. In the next step of the process, an imaging device 14 described above with respect to FIG. 1 is used to capture the light reflected from the retina 30.

After the image has been captured and stored, the image obtained is analyzed using a processor 16. For example, the processor 16 running NIH Image software, in an exemplary embodiment, may acquire, display, edit, enhance, analyze or animate images, or perform any combination of these functions. The software reads and writes TIFF, PICT, PICS and MacPaint files, providing compatibility with many other applications, including programs for scanning, processing, editing, publishing and analyzing images. The processor 16 running the exemplary NIH Image software supports many standard image processing functions, including contrast enhancement, density profiling, smoothing, sharpening, edge detection, median filtering, or spatial convolution with user defined kernels, or any combination of these functions.

The processor 16 running NIH Image can measure an area, mean, centroid, perimeter, etc. of user defined regions of interest. Automated particle analysis and tools for measuring path lengths and angles are also provided in the software. Spatial calibration is supported by the software to provide real world area and length measurements. Density calibration can be done against radiation or optical density standards using user specified units. Results can be printed, exported to text files, or copied to a clipboard program. The functional capabilities of NIH Image enable vascular and non-vascular tissue to be distinguished from each other, as well as arteries and veins. NIH Image also enables measurements of vasculature characteristics to be obtained from the pixel data using a variety of tools.

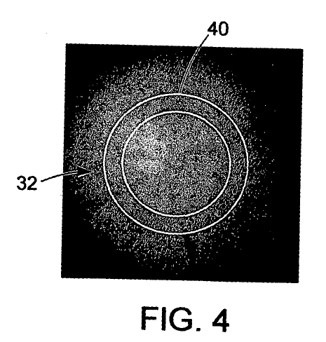

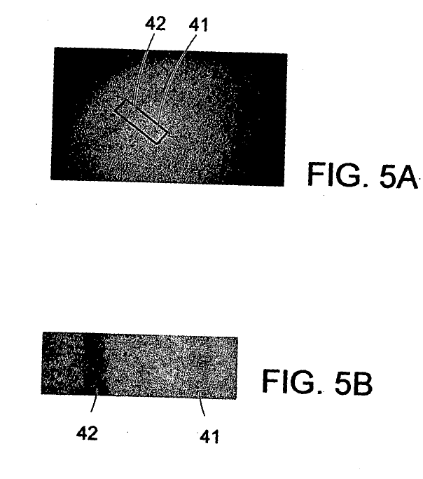

FIG. 4 is an image of the eye being analyzed with a custom software package. In the exemplary embodiment of the method of the invention, a medical professional draws a toroid 40 in the region of the optical disk 32 and the vasculature in the region of the optical disk 32 is analyzed. Spectroscopy as described above is used to differentiate between vascular and non-vascular tissue and/or between arteries veins and non vascular tissue in the toroid 40. For example, FIGS. 5A and 5B show the appearance of arteries, veins, and non vasculature tissue when illuminated by a light source 12 with a center frequency of about 570 nm and illustrate how spectroscopy can be used to distinguish arteries veins, and non vascular tissue. Retinal vessels appear darker than non-vascular retinal tissue. Arteries 41 appear lighter and more reflective, distinguishing them from veins 42, which appear darker as seen in the images obtained by the system 10 according to the invention. The contrast between arteries veins and non vascular tissue seen in FIGS. 5A and 5B results because of the different oxygenation levels of hemoglobin in the arteries and veins, which causes the arteries and veins to absorb and reflect light with varying intensities. This contrast can be detected by the processor 16 running, for example, NIH Image.

Once the arteries and veins have been distinguished through spectroscopy, a medical professional calculates the vascular volume, for example, by measurements of vessel diameter, to determine if the patient is at risk of experiencing vascular collapse. In one embodiment, measurements of diameters of these vessels may be obtained by taking a pixel count through a cross section of each vessel with the aid of a user defined area on the retina, for example a toroid 40 or a circle. For instance, with reference to FIG. 4, a medical professional may obtain the diameters of at least one artery, preferably all arteries, and at least one vein, preferably all veins, observed throughout the toroid 40 by taking pixel counts across a cross section of each of these vessels. Alternatively, measurements of the diameter of arteries and veins may be obtained on the border of the toroid 40 by taking pixel counts. In another embodiment, the medical professional obtains measurements of vessels, for example, arterial and/or venous diameter at any location of the eye of his choosing, and from as many arteries and veins as desired. In yet another embodiment, pixel count is made of all vascular tissue in the toroid 40, as well as a pixel count of all non vascular tissue in the toroid 40.

Once the desired arterial and venous measurements, for example diameter or area, are obtained, or once measurements of vascular and non vascular tissue have been made the data may be analyzed utilizing any number of techniques. For instance, in one embodiment, a ratio of venous diameter to arterial diameter is obtained, for example, for all arteries and veins passing through the toroid 40. To determine this ratio, the sum of the diameters of all the measured arteries is determined by a pixel count. The same process is conducted to obtain the sum of the diameters of all measured veins. A ratio of venous diameter to arterial diameter may then be determined by dividing the number of pixels representing the sum of venous diameter by the number of pixels representing the sum of arterial diameter. In another embodiment, a ratio of vascular area to non vascular area may be obtained by dividing the number of pixels representing vascular tissue in the toroid 40 by the number of pixels that depict non vascular tissue in the toroid 40.

According to an exemplary embodiment of the method of the invention, the medical professional compares the calculated measurements with a database to determine if the patient is at risk of vascular collapse. The database may include specific data from the patient obtained prior to the injury or after the injury, or data from other individuals with a known risk of vascular collapse. For instance, the ratio of venous diameter to arterial diameter calculated after the patient is injured is compared with the patient data that was obtained prior to the injury. If the ratio of venous diameter to arterial diameter after a surgical procedure, for example, differs from the ratio of venous diameter to arterial diameter for the same patient before a surgical procedure by a certain level, for instance 20%, medical staff will be alerted to provide care immediately because the patient may be at risk of hypovolemic shock. An alternate embodiment would provide an analog display, for example, a display reading from 0 to 5, where 0 represents normal blood volume and 5 represents significant blood loss requiring immediate treatment. A similar embodiment could include green, yellow, and red LEDs, for which illuminating a green LED would indicate normal blood volume, illuminating a yellow LED would indicate reduced but still adequate blood volume, and illuminating a red LED would indicate that immediate treatment was required.

In another embodiment, the ratio of venous diameter to arterial diameter from a patient may be compared to a database, for example, containing ratios obtained from individuals with known risk of vascular collapse to determine the patient's risk of vascular collapse. This technique is useful if patient specific data prior to injury is unavailable, perhaps because the patient is the victim of an accident. For example, comparing the ratio of venous diameter to arterial diameter of the patient to a database of known values for individuals at risk for vascular collapse will alert medical staff to intervene if the ratio of venous to arterial diameter obtained from the patient differs from, is below, or is above a predetermined range of acceptable ratio values, or is substantially similar to ratios obtained from individuals at risk for vascular collapse.

In another embodiment, the database may hold patient specific data obtained from a first image or set of images acquired after the injury has occurred. The database information may be compared to measurements obtained from a second image or set of images captured some time after the first image to follow the patient's condition and determine whether intervention is necessary. For instance, a first image of the patient's retinal vasculature may be obtained shortly after the trauma has occurred. After a prescribed amount of time has passed, for example, 10 to 15 minutes, a second image of the patient's eye may be taken as described above. Arteries, veins, and non vascular tissue may be distinguished and measurements made as described above and for example, venous to arterial ratios may be calculated for the first and second set of images. If the ratio of venous to arterial diameter differs in the two sets of images by a prescribed amount, medical staff may be alerted to intervene because the patient is at risk of suffering hypovolemic shock.

It will be appreciated that in addition to calculating a ratio of venous diameter to arterial diameter, any other calculation that aids detection of blood volume loss through measurement of retinal vasculature may be made in accordance with the invention. For instance, a sum of artery diameters may be obtained by a pixel count as described above, and this sum may be compared with patient specific data on artery diameter obtained before the injury. Alternatively, the sum of arterial diameter may be compared to a database of arterial diameters for individuals with a known risk of vascular collapse, or to patient specific data obtained shortly after the trauma has occurred. Likewise, an aggregate of venous diameter may be obtained and analyzed using the same techniques. In another embodiment, a light source 12 with a center wavelength, for example, of about 400 nm is used to distinguish retinal vasculature from non-vascular tissue and an aggregate measurement of retinal vascular tissue and an aggregate measurement of non vascular tissue are obtained by pixel count as described above. A ratio of retinal vascular tissue to non-retinal vascular tissue is then calculated by dividing the number of pixels representing retinal vasculature by the number of pixels representing non-retinal vasculature. This ratio is then compared to similar patient specific data obtained from the same location in the eye before or after the injury, or to a database of measurements of individuals with a known risk of vascular collapse to determine if the patient is at risk of vascular collapse. The above are examples of how the system 10 can be used to detect patients at risk of vascular collapse. It will be appreciated that other measurements and comparisons may be made using the system 10 to detect patients at risk of vascular collapse without exceeding the scope of the invention.

The following examples will serve to better demonstrate the successful practice of the present invention.

### EXEMPLIFICATION

A series of experiments were performed on rodents using the methods according to the invention to observe the effects of withdrawing a large volume of blood on retinal vasculature. In particular, the system according to FIG. 1 was used in the experiments. The rodents were anaesthetized using approximately 1500 mg/kg of urethane (ethyl carbamate) as an anesthetic. Urethane provided excellent analgesia during catheterization, good cycloplegia, and only minimally affected the cerebral hemodynamics. Tropicamide was applied topically to the corneal surface as needed to provide mydriasis and to augment the cycloplegia induced by the urethane. Mydriasis expanded the pupil enough to acquire retinal imagery, and cycloplegia greatly simplified the measurements.

### Experiment 1

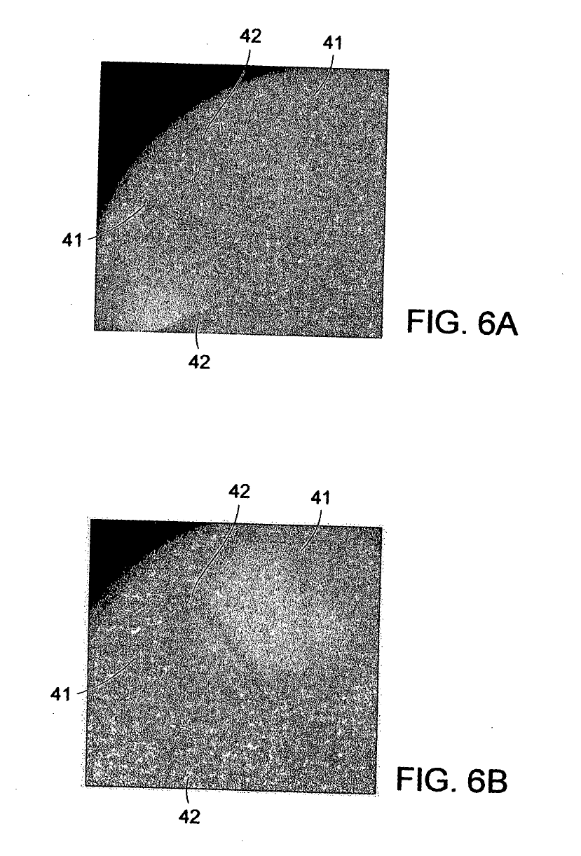

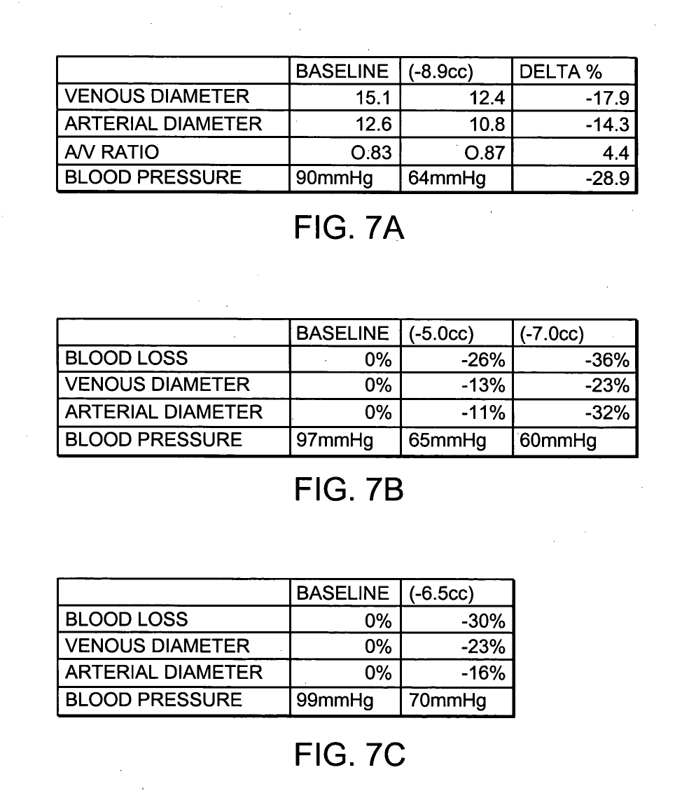

In the first experiment, baseline measurements of retinal and arterial diameter were obtained by measuring the diameters of arteries and veins prior to withdrawing blood from the rodent. The diameters of two arteries and two veins were obtained by taking a pixel count at cross sections of the two arteries and two veins shown in FIG. 6A. The sum of pixels for the two veins was found to be 15.1 pixels, and the sum of arterial pixels was found to be 12.6 pixels (FIG. 7A). An initial mean arterial blood pressure of 90 mmHg was measured with a piezoresistive pressure transducer at the distal end of a catheter placed in the right femoral artery.

After obtaining these measurements, 8.9 milliliters of blood was withdrawn from the rodent over a period of about 30 minutes, which represents approximately 32% of the rodent's normal circulating blood volume. Following the withdrawal, the rodent's blood pressure fell to 64 mmHg. Measurements of arterial and venous diameter were obtained on the same two veins and two arteries as described above (FIG. 6B) and at the same cross sectional locations. A venous diameter of 12.4 pixels and an arterial diameter of 10.8 pixels was measured. These values represent a decrease of 17.9% with respect to venous diameter, and a decrease of 14.3% with respect to arterial diameter (Column 3 of FIG. 7A). Ratios of arterial diameter to venous diameter were also calculated from the above data, revealing a ratio of 0.83 prior to the withdrawal of 8.9 milliliters of blood, and a ratio of 0.87 after the withdrawal of blood, representing an increase of 4.4%.

### Experiment 2

In a second experiment, which was conducted using the same set-up as described above for experiment one, the impact of both blood loss and blood pressure on retinal vasculature was examined. The results of the experiment are shown in FIG. 7B. A baseline mean arterial pressure of 97 mmHg was obtained after the rodent was anaesthetized and catheterized, but before any blood was withdrawn. Further, baseline measurements of arterial and venous diameter were obtained for two arteries and two veins using the same procedure as described in Experiment 1. Following these observations, 5.0 milliliters of blood were withdrawn from the rodent, representing a blood volume loss of 26%. After the blood withdrawal, the rodent's mean arterial pressure dropped to 65 mmHg (FIG. 7B, Column 2). Further, measurements of arterial and retinal diameter obtained as described above in Experiment 1 revealed a decrease of 11% in arterial diameter and a decrease of 13% in venous diameter from the baseline measurements. In the next step of the second experiment, a further 2 milliliters of blood were withdrawn from the rodent, representing a total blood volume loss of 36% from baseline conditions. At this volume of blood loss, measurements of arterial and venous diameter measured as described above decreased by 32% and 23% respectively from the baseline, and a mean arterial pressure of 60 mmHg was observed. The second experiment suggests that blood volume loss as the rodent nears vascular collapse impacts retinal vasculature more substantially than a decrease in blood pressure, since the mean arterial pressure decreased by 7.7% due to the additional withdrawal of 2 milliliters of blood, while arterial diameter and venous diameter decreased by 21% and 10%, respectively.

### Experiment 3

FIG. 7C depicts the results of the third experiment. In the third experiment, which was conducted using the same set-up as Experiments 1 and 2, baseline measurements of arterial and venous diameter were obtained by measuring the diameters of two arteries and two veins as described above, and an initial blood pressure of 99 mmHg was observed. A blood volume of 6.5 milliliters was then withdrawn from the rodent, and a blood pressure of 70 mmHg was observed. At this volume of blood loss, arterial diameter and venous diameter decreased by 16% and 23% from their respective baseline measurements. The three rodent experiments described suggest that a blood volume loss between about 20% and about 40% can be detected through measurements of retinal vessel diameter.

Other embodiments incorporating the concepts disclosed herein may be used without departing from the spirit and scope of the invention. The described embodiments are to be considered in all respects as only illustrative and not restrictive.

What is claimed is:

1. A system for assessing blood volume of a patient, comprising:
an imaging device for capturing images of the patient's retina as pixel data; and
a processor in communication with the imaging device, wherein the processor compares the pixel data to a database to determine if the patient is at risk of vascular collapse.

2. The system according to claim 1, wherein the processor measures a vasculature characteristic from the pixel data.

3. The system of claim 1 wherein the processor measures non-vascular tissue from the pixel data.

4. The system according to claim 1, wherein the imaging device captures images of the retina from a region around the patient's optical disk.

5. The system of claim 2 wherein the vasculature measurement comprises measuring arterial vessel diameter.

6. The system of claim 2 wherein the vasculature measurement comprises measuring venous vessel diameter.

7. The system of claim 2 wherein the vasculature measurement comprises measurements of arterial and venous vessel diameters.

8. The system of claim 7, wherein a ratio of venous diameter to arterial diameter is calculated from the pixel data, and the ratio is compared to the database to determine if the patient is at risk of vascular collapse.

9. The system of claim 1 wherein the pixel data is obtained from a user-defined area on the retina.

10. The system according to claim 9 wherein the user-defined area is toroidal in shape.

11. The system according to claim 9 wherein the user-defined area is circular in shape.

12. The system according to claim 1 wherein the imaging device comprises a CCD-based camera for capturing images of the patient's retina.

13. The system according to claim 1 wherein the imaging device comprises a MOS based camera for capturing images of the patient's retina.

14. The system according to claim 1 wherein the imaging device comprises a single element detector.

15. The system according to claim 2 or 3 wherein the processor outputs an alert if the measurements are below or above a predetermined range of values.

16. The system of claim 1 wherein the processor distinguishes between vascular and non vascular tissues.

17. The system of claim 1, wherein the processor distinguishes between arterial vessels, venous vessels, and non-vascular tissues.

18. The system of claim 1 further comprising an output device.

19. The system of claim 18, wherein the output device is selected from a laptop monitor, a desktop computer monitor, a television screen, a PDA, a printing device, and a pager.

20. The system according to claim 1 further comprising a light source.

21. The system according to claim 20, wherein the light source is selected from the group consisting of a light emitting diode, an incandescent light bulb, a flash lamp, and a laser diode.

22. The system of claim 1 wherein the data is captured at a center wavelength in the range of about 400 nm to about 1000 nm.

23. The system of claim 1 wherein the data is captured at a center wavelength in the range of about 500 nm to 700 nm.

24. The system according to claim 20 wherein the light source provides light having a center wavelength in the range of about 400 nm to about 1000 nm.

25. The system according to claim 20 wherein the light source provides light having a center wavelength in the range of about 500 nm to about 700 nm.

26. The system of claim 1 further comprising an optical assembly.

27. The system of claim 1, wherein the system is portable.

28. A method for assessing blood volume of a patient, comprising:
capturing images of the patient's retina as pixel data using an imaging device;
using a processor to calculate measurements of retinal vessels from the pixel data; and
comparing the calculated measurements with a database to determine if the patient is at risk of vascular collapse.

29. A method according to claim 28 further comprising the step of outputting an alert if the measurements are below or above a predetermined range of values.

30. The method of claim 28 further comprising the step of using spectrometry to distinguish arterial vessels, venous vessels and non-vascular tissue.

31. The method of claim 28 wherein the step of comparing the calculated measurements with a database to determine if the patient is at risk of vascular collapse further comprises using a database comprising patient specific data obtained from the patient before or after the injury, or data from individuals with a known risk of vascular collapse.
